(19) United States
(12) Patent Application Publication
(10) Pub. No.: US 2010/0121418 A1
(43) Pub. Date: May 13, 2010

(54) SKIN COOLING SYSTEM

(76) Inventors: Dae Sic LEE, Ansan-si (KR); Guang Hoon Kim, Busan (KR); Uk Kang, Ansan-si (KR); R. Andreev, Saint Petersburg (RU)

Correspondence Address: FROMMER LAWRENCE & HAUG LLP, 745 FIFTH AVENUE, NEW YORK, NY, 10151, US

(21) Appl. No.: 12/474,522

(22) Filed: May 29, 2009

(30) Foreign Application Priority Data
Nov. 13, 2008 (KR) 10-2008-0112557

(51) Int. Cl. A61N 5/067; A61F 7/00

(52) U.S. Cl. 607/89; 607/104

(57) ABSTRACT

The present invention provides a skin cooling system which can improve skin cooling efficiency and significantly reduce thermal damage and pain caused by heat generated during treatment, using a handpiece for injecting cold air, water, or a mixture of cold air and water.

For this purpose, the present invention provides a skin cooling system including: a handpiece for focusing therapeutic light energy onto the surface of the skin for treatment and injecting cold air, water, or a mixture of cold air and water to the skin during treatment; a cold air supply means connected to the handpiece to supply cold air; and a cold water supply means connected to the handpiece to supply cooling water.

### CROSS-REFERENCE TO RELATED APPLICATION

This application claims the benefit of Korean Patent Application No. 10-2008-0112557, filed on Nov. 13, 2008, the entire disclosure of which is hereby incorporated by reference.

### BACKGOUND OF THE INVENTION

1. Field of the Invention

The present invention relates to a skin cooling system, and more particularly, to a skin cooling system which can improve skin cooling efficiency and significantly reduce thermal damage and pain caused by heat generated during treatment using a handpiece for injecting cold air, water, or a mixture of cold air and water.

2. Description of Related Art

In general, medical devices used in various skin treatments such as medical laser devices, high frequency systems, etc. are used to treat skin diseases by applying heat to the skin. Such medical devices generate heat by conversion of light or vibration energy during operation to treat the skin diseases. The heat is used to treat the object such as skin.

As such, the skin diseases are treated using the heat generated by the conversion of light energy; however, when a large amount of heat is accumulated in the skin during treatment, it may cause skin damage. Thus, the skin is cooled before or during treatment to prevent the thermal damage, thereby reducing skin damage and pain caused by the heat.

Methods of cooling the skin during treatment include a cooling method using cold air, a contact cooling method, a vaporization cooling method using cryogen, an ice pack or ice roller cooling method using ice, and the like.

However, the conventional methods of cooling the skin during treatment have the following problems.

The cooling method using cold air cools the skin during treatment by applying cold air to the surface of the skin. However, the capability of transmitting the temperature is very low, which results in a low cooling efficiency and a long cooling time.

The contact cooling method also has problems in that the configuration of a handpiece tip, a device for treating skin diseases by contacting the skin, is limited, it is difficult to uniformly contact the surface of the skin during treatment, and thus the cooling efficiency is limited.

The vaporization cooling method can cool the skin for a short time; however, the cooling retention time is very short, and it may cause frostbite on exposed skin.

The ice pack or ice roller cooling method also has problems in that the ice pack or ice roller used to cool the skin should be frequently changed, which requires a long preparation time, and it is difficult to use the ice pack or ice roller during the treatment.

### SUMMARY OF THE INVENTION

The present invention has been made in an effort to solve the above-described problems associated with prior art. Accordingly, the present invention provides a skin cooling system, which can cool the skin during treatment for a short time, significantly reduce thermal damage and pain caused by heat generated by conversion of light energy during treatment, and prevent sequelae after treatment using a handpiece that injects cold air, water, or a mixture of cold air and water.

In one aspect, the present invention provides a skin cooling system including: a handpiece for focusing therapeutic light energy onto the surface of the skin for treatment and injecting cold air, water, or a mixture of cold air and water to the skin during treatment; a cold air supply means connected to the handpiece to supply cold air; and a water supply means connected to the handpiece to supply water.

In a preferred embodiment, the handpiece includes: a grip body; an optical unit mounted in the middle of the grip body and generating therapeutic light energy; a spray nozzle mounted on an inner side of the grip body and injecting cold air, water, or a mixture of cold air and water to the skin; a skin contact surface integrally formed at a lower end of the grip body; a working (cooling) chamber which is concavely upward in the center of the skin contact surface; a window mounted on the upper part of the working (cooling) chamber to protect the optical unit from foreign substances; a nozzle port formed on one side wall of the working (cooling) chamber and injecting cold air, water, or a mixture of cold air and water from the spray nozzle to the skin; and a connecting pipe provided on an inner side of the grip body to connect the spray nozzle and the nozzle port.

In another preferred embodiment, the handpiece further includes: an intake port formed on the other side wall of the working (cooling) chamber to suck the water injected to the surface of the skin through the nozzle port and foreign substances generated during treatment; and an intake pipe connected to the intake port and mounted on the other inner side of the grip body.

In still another preferred embodiment, an outlet of the intake pipe is connected to a water/foreign substance remover for providing a vacuum suction force.

In yet another preferred embodiment, the cold air supply means for supplying the cold air to the hand-piece includes: a dust filter for removing foreign substances contained in the air; an air compressor for compressing air; an air tank for storing the compressed air at a predetermined pressure; a regulator for regulating the pressure of the air discharged from the air tank and discharging the pressure-regulated air; a water/oil remover for removing water and oil in the discharged air; an air cooler for cooling the dry air to a predetermined temperature; a first valve connected to an outlet of the air cooler; a flow meter for controlling the amount of cold air discharged through the first valve; and a first connecting line for connecting an outlet of the flow meter and the spray nozzle of the handpiece.

In still yet another preferred embodiment, the water supply means for supplying cold water to the hand-piece includes: a water tank having a predetermined volume; a pump for supplying water from the water tank; a water cooler for cooling the water supplied by the pump to a predetermined temperature; a second valve connected to an outlet of the water cooler; and a second connecting line for connecting an outlet of the second valve and the spray nozzle of the handpiece.

### BRIEF DESCRIPTION OF THE DRAWINGS

The above and other features of the present invention will be described with reference to certain exemplary embodiments thereof illustrated the attached drawings in which:

FIG. 1 is a diagram showing an overall configuration of a skin cooling system in accordance with the present invention; and

FIG. 2 is a schematic cross-sectional view showing a handpiece, a main component of the skin cooling system in accordance with the present invention.

### DETAILED DESCRIPTION OF THE INVENTION

Hereinafter, preferred embodiments of the present invention will be described in detail with reference to the accompanying drawings.

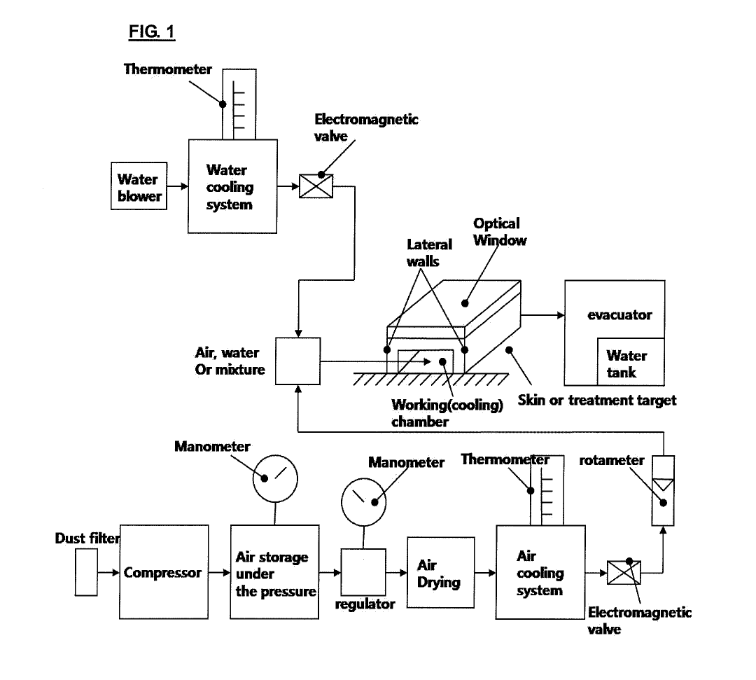

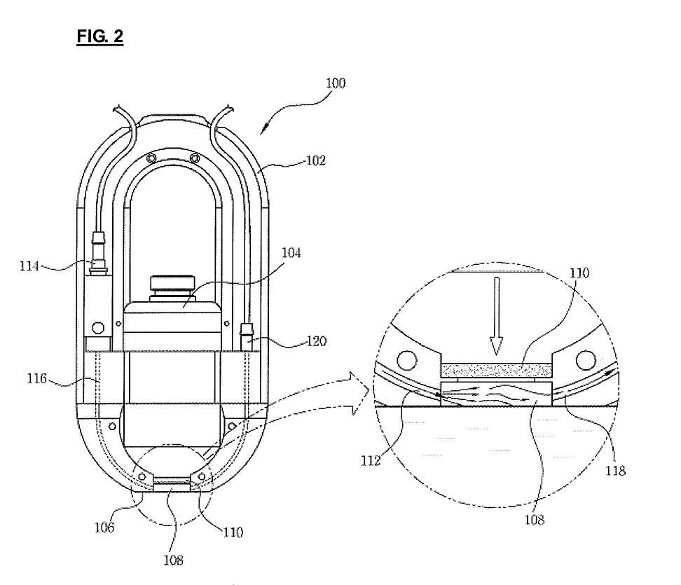

FIG. 1 is a diagram showing an overall configuration of a skin cooling system in accordance with the present invention, and FIG. 2 is a schematic cross-sectional view showing a handpiece, a main component of the skin cooling system in accordance with the present invention.

The skin cooling system in accordance with the present invention includes a handpiece as a main component having the function of focusing therapeutic light energy (e.g., laser beam) onto the skin and generating heat, and the function of injecting cold air, water, or a mixture of cold air and water to the skin for cooling during treatment. The handpiece is connected to a cold air supply means for supplying cold air and a water supply means for supplying cooling water.

The configuration of the handpiece will be described in more detail below.

The handpiece 100 is a component which will is held in an operator's hand and brought into contact with the skin for treatment using therapeutic light energy, and includes a grip body 102 that an operator can hold.

An optical unit 104, which focuses the therapeutic light energy, i.e., laser beam, onto the skin and transmits the light energy to the skin is mounted in the middle of the inside of the grip body 102 of the handpiece 100.

Moreover, a skin contact surface 106 having a horizontal surface which will be brought into direct contact with the skin is integrally formed at a lower end of the grip body 102 of the handpiece 100. A working (cooling) chamber 108 which is concave upward is placed in the center of the skin contact surface 106.

A window 110 for preventing foreign substances generated during skin treatment from being introduced into the optical unit 104 is integrally mounted on the upper part of the working (cooling) chamber 108.

Especially, a nozzle port 112, through which cold air, water, or a mixture of cold air and water is injected to the skin, is provided on one side wall of the working (cooling) chamber 108.

Moreover, a spray nozzle 114 for injecting cold air, water, or a mixture of cold air and water to the skin through the nozzle port 112 is integrally mounted on an inner side of the grip body 102 of the handpiece 100, and the spray nozzle 114 and the nozzle port 112 are connected to each other by a connecting pipe 116.

The configuration of the cold air supply means connected to the spray nozzle of the handpiece to supply cold air to the handpiece will be described in detail below.

A dust filter 202 for removing foreign substances contained in the air and an air compressor 204 for compressing air are disposed in a front portion of the cold air supply means 200, and an air tank 206 for storing the compressed air at a predetermined pressure is located at an outlet of the air compressor 204.

A regulator 208 for regulating the pressure of the air discharged from the air tank 206 and discharging the pressure-regulated air is connected to an outlet of the air tank 206, and a water/oil remover 210 for removing water and oil in the air is located at an outlet of the regulator 208.

An air cooler 212 for cooling the dry air, from which water and oil are removed, to a predetermined temperature is located at an outlet of the water/oil remover 210. The dry air is cooled to a predetermined temperature by a vortex tube in the air cooler 212.

A first valve 214, a kind of electromagnetic solenoid valve, is connected to an outlet of the air cooler 212, and a flow meter 216 for controlling the optional amount of cold air discharged is connected to an outlet of the first valve 214.

Here, the outlet of the flow meter 216 and the spray nozzle 114 of the handpiece 100 are connected to each other by a first connecting line 218.

Next, the configuration of the water supply means connected to the spray nozzle of the handpiece to supply cooling water to the handpiece will be described in detail.

The water supply means 300 includes a water tank 302 having a predetermined volume and containing water for cooling the skin, a pump 304 connected to an outlet of the water tank 302 and supplying water, a water cooler 306 for cooling the water supplied by the pump 304 to a predetermined temperature, and a second valve 308, a kind of electromagnetic solenoid valve, connected to an outlet of the water cooler 306.

Here, the outlet of the second valve 308 and the spray nozzle 114 of the handpiece 100 are connected to each other by a second connecting line 310.

Meanwhile, the handpiece 100 further includes a means for absorbing and removing water injected to the skin and foreign substances generated during the skin treatment in real time.

That is, an intake port 118 for sucking the water injected to the surface of the skin and the foreign substances generated during treatment is provided on the other side wall of the working (cooling) chamber 108 in the skin contact surface 106 of the handpiece 100. Further, an intake pipe 120 connected to the intake port 118 is mounted on the other inner side of the grip body 102 of the handpiece 100.

A water/foreign substance remover 122 for providing a vacuum suction force is connected to an outlet of the intake pipe 120.

Next, an operation example of the skin cooling system having the above-described configuration in accordance with the present invention will be described.

When the skin contact surface 106 of the handpiece 100 held in an operator's hand is brought into contact with the skin for treatment, the therapeutic light energy radiated from the optical unit 104 is focused onto the surface of the skin for treatment through the window 110.

As the time the therapeutic light energy is transmitted to the skin continues, heat is generated, which may cause damage to the skin.

At this time, the skin may be cooled by low-temperature cold air, low-temperature cooling water, or a mixture of cold air and water.

First, for the cooling process by the cold air, the first valve 214 is opened and, in this case, the opening and closing of the first valve 214 may be controlled by a controller (not shown) according to the operation of a switch (not shown).

Accordingly, the outside air, from which foreign substances are removed by the dust filter 202, is compressed by the air compressor 204 and stored in the air tank 206 at a predetermined pressure. Then, the pressure of the air discharged from the air tank 206 is regulated by the regulator 208 connected to the outlet of the air tank 206, and the pressure-regulated air is discharged to the water/oil remover 210. Subsequently, the water and oil contained in the discharged air is removed by the water/oil remover 210, and the resulting air is cooled to a predetermined temperature in the air cooler 212. Next, the cold air passes through the first valve 214 and is then supplied to the spray nozzle 114 of the handpiece 100 through the first connecting line 218.

At the same time, the cold air supplied to the spray nozzle 114 of the handpiece 100 flows toward the nozzle port 112 formed on one side wall of the working (cooling) chamber 108 through the connecting pipe 116 and is then injected to the surface of the skin during treatment through the nozzle port 112.

The skin can be easily cooled by the cold air injected to the surface of the skin, and thus it is possible to significantly reduce skin damage and pain caused by the heat generated by the therapeutic light energy.

Meanwhile, to cool the skin using the cooling water, not the cold air, the operator closes the first valve 214 and opens the second valve 308. Also, the opening and closing of the second valve 308 may be controlled by a controller (not shown) according to the operation of a switch (not shown).

Accordingly, water is supplied from the water tank 302 to the water cooler 306 by the operation of the pump 304. The supplied water is cooled to a predetermined temperature in the water cooler 306. Then, the cooling water passes through the second valve 308 connected to the outlet of the water cooler 306 and is then supplied to the spray nozzle 114 of the handpiece 100.

At the same time, the cooling water supplied to the spray nozzle 114 of the handpiece 100 flows toward the nozzle port 112 formed on one side wall of the working (cooling) chamber 108 through the connecting pipe 116 and is then injected to the surface of the skin during treatment through the nozzle port 112.

In the same manner as the cold air, the skin can be easily cooled by the cooling water injected to the surface of the skin, and thus it is possible to significantly reduce skin damage and pain caused by the heat generated by the therapeutic light energy.

Here, when a mixture of cold air and water is injected, it is possible to effectively perform the skin cooling process for a shorter time and extend the cooling retention time of the skin.

That is, when the first valve 214 and the second valve 308 are simultaneously opened, the cold air and water supplied in the manner described above are mixed in the spray nozzle 114 of the handpiece 100 and injected to the surface of the skin during treatment through the nozzle port 112.

As such, since it is possible to effectively perform the skin cooling process for a shorter time and extend the cooling retention time of the skin by injecting a mixture of cold air and water to the surface of the skin, it is possible to more significantly reduce skin damage and pain caused by the heat generated by the therapeutic light energy.

Meanwhile, when the skin treatment is performed in a state where cold air, water, or a mixture of cold air and water is injected to the surface of the skin, the water injected to the surface of the skin and the foreign substances generated from the skin during the treatment are mixed and the mixture remains on the surface of the skin, which may disturb the skin treatment.

Thus, the water/foreign substance remover 122 is operated such that its vacuum suction force is transmitted to the intake port 118 provided on the other side wall of the working (cooling) chamber 108 in the skin contact surface 106 of the handpiece 100 through the intake pipe 120 mounted on the other inner side of the grip body 102 of the handpiece 100. Then, the water injected to the surface of the skin and the foreign substances generated from the skin during the treatment are absorbed and removed by the water/foreign substance remover 122 through the intake port 118 and the intake pipe 120.

As a result, the skin treatment can be effectively performed while cooling the skin and keeping the surface of the skin clean.

As described above, the present invention provides the following effects.

With the use of the handpiece, which is connected to the cold air supply means and the cold water supply means to inject cold air, water, or a mixture of cold air and water, it is possible to cool the skin during treatment for a short time and maintain the cooling effect during the treatment.

Thus, the skin cooling system of the present invention can easily cool the skin before or during treatment, and thus significantly reduce thermal damage and pain caused by the heat generated by the therapeutic light energy during treatment.

As above, preferred embodiments of the present invention have been described and illustrated, however, the present invention is not limited thereto, rather, it should be understood that various modifications and variations of the present invention can be made thereto by those skilled in the art without departing from the spirit and the technical scope of the present invention as defined by the appended claims.

What is claimed is:

1. A skin cooling system comprising:
a handpiece for focusing therapeutic light energy onto the surface of the skin for treatment and injecting cold air, water, or a mixture of cold air and water to the skin during treatment;
a cold air supply means connected to the handpiece to supply cold air; and
a water supply means connected to the handpiece to supply cooling water.

2. The system of claim 1, wherein the handpiece comprises:
a grip body;
an optical unit mounted in the middle of the grip body and generating therapeutic light energy;
a spray nozzle mounted on an inner side of the grip body and injecting cold air, water, or a mixture of cold air and water to the skin;
a skin contact surface integrally formed at a lower end of the grip body;
a working (cooling) chamber which is concave upward in the center of the skin contact surface;
a window mounted on the upper part of the working (cooling) chamber to protect the optical unit from foreign substances;
a nozzle port formed on one side wall of the working (cooling) chamber and injecting cold air, water, or a mixture of cold air and water from the spray nozzle to the skin; and
a connecting pipe provided on an inner side of the grip body to connect the spray nozzle and the nozzle port.

3. The system of claim 2, wherein the handpiece further comprises:
an intake port formed on the other side wall of the working (cooling) chamber to suck the water injected to the surface of the skin through the nozzle port and foreign substances generated during treatment; and
an intake pipe connected to the intake port and mounted on the other inner side of the grip body.

4. The system of claim 3, wherein an outlet of the intake pipe is connected to a water/foreign substance remover for providing a vacuum suction force.

5. The system of claim 1, wherein the cold air supply means connected for supplying the cold air to the hand-piece comprises:
a dust filter for removing foreign substances contained in the air;
an air compressor for compressing air;
an air tank for storing the compressed air at a predetermined pressure;
a regulator for regulating the pressure of the air discharged from the air tank and discharging the pressure-regulated air;
a water/oil remover for removing water and oil in the discharged air;
an air cooler for cooling the dry air to a predetermined temperature;
a first valve connected to an outlet of the air cooler;
a flow meter for controlling the amount of cold air discharged through the first valve; and
a first connecting line for connecting an outlet of the flow meter and the spray nozzle of the handpiece.

6. The system of claim 1, wherein the cold water supply means connected for supplying the cold water to the hand-piece comprises:
a water tank having a predetermined volume;
a pump for supplying water from the water tank;
a water cooler for cooling the water supplied by the pump to a predetermined temperature;
a second valve connected to an outlet of the water cooler; and
a second connecting line for connecting an outlet of the second valve and the spray nozzle of the handpiece.
